SEQUENCE LISTING (1 sequence)

SEQ ID NO: 1 - protein, 1409 aa, Homo sapiens
  TENC1
MKSSGPVERLLRALGRRDSSRAASRPRKAEPHSFREKVFRKKPPVCAVCKVTIDGTGVSCRVCKVATHRKCEAKVTSACQALPPVELRRNTAPVRRIEHLGSTKSLNHSKQRSTLPRSFSLDPLMERRWDLDLTYVTERILAAAFPARPDEQRHRGHLRELAHVLQSKHRDKYLLFNLSEKRHDLTRLNPKVQDFGWPELHAPPLDKLCSICKAMETWLSADPQHVVVLYCKGNKGKLGVIVSAYMHYSKISAGADQALATLTMRKFCEDKVATELQPSQRRYISYFSGLLSGSIRMNSSPLFLHYVLIPMLPAFEPGTGFQPFLKIYQSMQLVYTSGVYHIAGPGPQQLCISLEPALLLKGDVMVTCYHKGGRGTDRTLVFRVQFHTCTIHGPQLTFPKDQLDEAWTDERFPFQASVEFVFSSSPEKIKGSTPRNDPSVSVDYNTTEPAVRWDSYENFNQHHEDSVDGSLTHTRGPLDGSPYAQVQRPPRQTPPAPSPEPPPPPMLSVSSDSGHSSTLTTEPAAESPGRPPPTAAERQELDRLLGGCGVASGGRGAGRETAILDDEEQPTVGGGPHLGVYPGHRPGLSRHCSCRQGYREPCGVPNGGYYRPEGTLERRRLAYGGYEGSPQGYAEASMEKRRLCRSLSEGLYPYPPEMGKPATGDFGYRAPGYREVVILEDPGLPALYPCPACEEKLALPTAALYGLRLEREAGEGWASEAGKPLLHPVRPGHPLPLLLPACGHHHAPMPDYSCLKPPKAGEEGHEGCSYTMCPEGRYGHPGYPALVTYSYGGAVPSYCPAYGRVPHSCGSPGEGRGYPSPGAHSPRAGSISPGSPPYPQSRKLSYEIPTEEGGDRYPLPGHLASAGPLASAESLEPVSWREGPSGHSTLPRSPRDAPCSASSELSGPSTPLHTSSPVQGKESTRRQDTRSPTSAPTQRLSPGEALPPVSQAGTGKAPELPSGSGPEPLAPSPVSPTFPPSSPSDWPQERSPGGHSDGASPRSPVPTTLPGLRHAPWQGPRGPPDSPDGSPLTPVPSQMPWLVASPEPPQSSPTPAFPLAASYDTNGLSQPPLPEKRHLPGPGQQPGPWGPEQASSPARGISHHVTFAPLLSDNVPQTPEPPTQESQSNVKFVQDTSKFWYKPHLSRDQAIALLKDKDPGAFLIRDSHSFQGAYGLALKVATPPPSAQPWKGDPVEQLVRHFLIETGPKGVKIKGCPSEPYFGSLSALVSQHSISPISLPCCLRILSKDPLEETPEAPVPTNMSTAADLLRQGAACSVLYLTSVETESLTGPQAVARASSAALSCSPRPTPAVVHFKVSAQGITLTDNQRKLFFRRHYPVNSITFSSTDPQDRRWTNPDGTTSKIFGFVAKKPGSPWENVCHLFAELDPDQPAGAIVTFITKVLLGQRK

(19) United States
(12) Patent Application Publication
(10) Pub. No.: US 2016/0361288 A1
(43) Pub. Date: Dec. 15, 2016

(54) PHARMACEUTICAL COMPOSITIONS FOR PREVENTING OR TREATING DIABETIC NEPHROPATHY COMPRISING THE ACTIVITY INHIBITOR OF TENC1

(71) Applicant: POSTECH ACADEMY-INDUSTRY FOUNDATION, Pohang-si (KR)

(72) Inventors: Sung Ho RYU, Pohang-si (KR); Jiyoun LEE, Pohang-si (KR); Heeyoon JEONG, Pohang-si (KR); Ara KOH, Seoul (KR)

(73) Assignee: POSTECH ACADEMY-INDUSTRY FOUNDATION, Pohang-si (KR)

(21) Appl. No.: 15/176,064

(22) Filed: Jun. 7, 2016

(30) Foreign Application Priority Data
Jun. 10, 2015 (KR) 10-2015-0081917
May 31, 2016 (KR) 10-2016-0066958

(51) Int. Cl. A61K 31/343

(52) U.S. Cl. CPC A61K 31/343

(57) ABSTRACT

Provided is a pharmaceutical composition for preventing or treating diabetic nephropathy, comprising a tensin like C1 domain containing phosphatase (TENC1) inhibitor as an active ingredient. A new target for treating diabetic nephropathy is presented by confirming that TENC1 expression is increased in kidney tissue of diabetes or a podocyte cell line to which a high blood glucose environment is given and experimentally proving that nephrin phosphorylation inhibited by the PTPase activity of TENC1 affects the permeability and mTORC1 signaling of the podocytes resulting in inducing podocyte hypertrophy. As the pharmaceutical composition comprising a TENC1 inhibitor as an active ingredient inhibits nephrin dephosphorylation by TENC1, podocytes damaged from an early stage of the diabetic nephropathy may be protected and the structure and filtration function of the podocytes may be maintained, therefore the pharmaceutical composition is expected to be widely used in preventing or treating the diabetic nephropathy from an early stage.

### CROSS-REFERENCE TO RELATED APPLICATION

This application claims priority to and the benefit of Korean Patent Application No. 10-2015-0081917, filed on Jun. 10, 2015, and Korean Patent Application No. 10-2016-0066958, filed on May 31, 2016, the disclosures of which are incorporated herein by reference in its entirety.

### TECHNICAL FIELD

The present invention relates to a pharmaceutical composition for preventing or treating diabetic nephropathy comprising a tensin-like C1 domain containing phosphatase (TENC1) inhibitor as an active ingredient.

### BACKGROUND ART

As the aging society continues, metabolic diseases including diabetes are gradually on the rise. Diabetes is a representative metabolic disease which is an event of a high blood glucose concentration caused by abnormal secretion of insulin from a pancreas or reduction of insulin reactivity at peripheral regions because of a problem in insulin signaling. Recently, in Korea there is also an increase of diabetic population due to social and economic development. In 2012, Korean Diabetes Association reported that, in Korean population, about 3,200,000 adults corresponding to one out of ten adults have been diagnosed with diabetes and two out of ten adults are prediabetic. If this trend continues, by year 2050, diabetic patients in Korea is estimated to reach 6 millions. Also, only 73.4% of all the diabetic patients are aware of having the disease, and, particularly, approximately half (45.6%) of the young patients between 30 to 44 are not even aware of having the disease and thus run a high risk of being exposed to diabetic complications due to delayed treatment.

Diabetic nephropathy (DN) which is one of the main complications of diabetes along with retinopathy and neuropathy develops in 20 to 40% of the diabetic patients and has been recently reported as the main cause of end-stage renal disease (ESRD) all over the world including Korea because of increasing diabetic patients. When a patient with diabetic nephropathy reaches ESRD, the patient's kidney is irreversibly damaged. Since there is no effective prevention and treatment method for such damage, only renal replacement therapy such as chronic hemodialysis or peritoneal dialysis is available until renal transplantation. This therapy significantly diminishes the quality of a patient's life and requires an astronomical increase of medical expenses due to continuous treatment. Accordingly, it is very important that diabetic nephropathy is recognized and treated in an early stage to prevent ESRD, before renal fibrosis develops.

Most of conventional research on diabetic nephropathy focused on glomerulosclerosis caused by growth and hypertrophy of mesangium or fibrosis caused by accumulation of extracellular matrix in renal tubular interstitium.

Meanwhile, podocytes are cells of glomerular filtration barrier constituting the outermost layer and filters blood in capillaries of the glomerulus through the urine filtration system to form urine and play an important role in maintaining a normal structure and the urine filtration system of the kidney. It has been known that the podocytes are damaged from the early stage of diabetic nephropathy and may be caused by a decrease in the number of the podocytes, a structural change and a functional damage. In the early stage of the diabetic nephropathy, while there is no change in the number of other cells in the glomerulus, the decrease in the number of the podocytes is observed, and foot process effacement, an increase in the width of the foot process, and a structural change such as decreased density of the slit diaphragm (SD) are observed. It is known that, in diabetes, high glucose, an advanced glycation end-product (AGE), angiotensin II, physical cell stretching associated with high blood pressure or an increase of intravesical pressure, reactive oxygen species (ROS), and a transforming growth factor (TGF) β1, etc. may be damage mechanisms on podocytes. Therefore, the observation/understanding of the changes in podocytes in the diabetic nephropathy may be very important in early diagnosis, prevention and treatment of the diabetic nephropathy. However, the research trend on such damage mechanisms does not stray far from the conventional research framework of the mesangium which is another glomerulus cell, and thus it is necessary to research the podocyte damage mechanism in a new way.

Nephrin which constitutes the slit diaphragm is a transmembrane protein of the podocyte. The extracellular domain of nephrin is in interaction on neighboring podocyte foot processes and thus plays a key role in forming the slit diaphragm, and the intracellular domain of nephrin is in connection with a proteins such as CD2AP, Neph1 or podocin in cells and serves to maintain the structure of the podocyte. As it was identified that mutation of a gene for nephrin causes congenital nephrotic syndrome of the Finnish type showing severe proteinuria and nephrotic syndrome from a fetus, the importance of nephrin in the glomerular filtration mechanism started receiving limelight. Recently, it was presented that nephrin plays important role in the podocyte signaling cascade through phosphorylation of a tyrosine residues of the nephrin intracellular domain by Fyn, which is a src-kinase family. Until now, research has focused on the maintaining the podocyte structure through controlling an actin cytoskeleton via the phosphorylated nephrin which is recognized by a non-catalytic region of tyrosine kinase adaptor protein 1 (Nck) having a Src homology 2 (SH2) domain or phosphoinositide 3-kinase (PI3K). However, research on downstream signaling in the podocyte via nephrin phosphorylation and its controller is still not enough. Particularly, although research on the reduction of nephrin phosphorylation in damaged kidneys has been reported, it is not well understood about a protein tyrosine phosphatase (PTPase) mediating nephrin dephosphorylation, and therefore research for understanding nephrin dephosphorylation-mediated podocyte damage and mechanisms of kidney damage is needed.

### DISCLOSURE

### Technical Problem

To solve the above-described conventional problems, the inventors have conducted research on a podocyte damage caused by the dephosphorylation of nephrin in the early stage of diabetic nephropathy, and further the damage mechanisms on the kidney from understanding that tensin-like C1 domain-containing phosphatase (TENC1) having a PTPase activity is highly expressed in the diabetic kidney, and thereby found that nephrin dephosphorylation is mediated by TENC1 in diabetic nephropathy, leading to the completion of the present invention.

Therefore, the present invention is directed to providing a pharmaceutical composition which comprises a TENC1 inhibitor as an active ingredient for preventing or treating diabetic nephropathy.

However, the technical problems to be solved by the present invention are not limited to the problems described above, and other problems which have not been described may be clearly understood to one of ordinary skill in the art from the following description.

### Technical Solution

To achieve the object of the present invention, in one aspect, the present invention provides a pharmaceutical composition for preventing or treating diabetic nephropathy, which comprises a TENC1 inhibitor as an active ingredient.

In an exemplary embodiment of the present invention, the TENC1 inhibitor may be a naphthoquinone-based compound.

In another exemplary embodiment of the present invention, the naphthoquinone-based compound may be one or more selected from the group consisting of (1R)-1,6-dimethyl-1,2-dihydronaphtho[1,2-g][1]benzofuran-10,11-dione (dihydrotanshinone; DHTS), (1R)-1,6,6-trimethyl-2,7,8,9-tetrahydro-1H-naphtho[1,2-g][1]benzofuran-10,11-dione (cryptotanshinone), 2,2-dimethyl-3,4-dihydrobenzo[h]chromene-5,6-dione (β-lapachone), and (4Z)-5-amino-6-(7-amino-6-methoxy-5,8-dioxoquinolin-2-yl)-4-(4,5-dimethoxy-6-oxocyclohexa-2,4-dien-1-ylidene)-3-methyl-1H-pyridine-2-carboxylic acid (streptonigrin).

In still another exemplary embodiment of the present invention, the naphthoquinone-based compound may be dihydrotanshinone.

In yet another exemplary embodiment of the present invention, the TENC1 may comprise the amino acid sequence of SEQ. ID. NO: 1.

In yet another exemplary embodiment of the present invention, the TENC1 inhibitor may inhibit a PTPase activity of TENC1.

In yet another exemplary embodiment of the present invention, the composition may have a podocyte protective effect by inhibiting nephrin dephosphorylation by TENC1.

In yet another exemplary embodiment of the present invention, the composition may further comprise a pharmaceutically acceptable carrier or additive.

Also, in another aspect, the present invention provides a method for preventing or treating diabetic nephropathy, comprising administering the composition to a subject.

Also, in still another aspect, the present invention provides a use of the composition for preventing or treating diabetic nephropathy.

### Advantageous Effects

The present invention provides a new target for treating diabetic nephropathy by confirming that TENC1 expression is increased in kidney tissue of diabetes or a podocyte cell line to which a high blood glucose environment is given and experimentally proving that nephrin phosphorylation inhibited by the PTPase activity of TENC1 affects permeability and mTORC1 signaling of the podocytes resulting in inducing podocyte hypertrophy. Therefore, a pharmaceutical composition comprising a TENC1 inhibitor as an active ingredient according to the present invention inhibits nephrin dephosphorylation by TENC1, thereby protecting podocytes damaged from an early stage of the diabetic nephropathy and maintaining the structure and filtration function of the podocytes. Therefore, the pharmaceutical composition of the present invention is expected to be widely used in preventing or treating the diabetic nephropathy from an early stage.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 shows an increase in expression of TENC1 protein in the kidney of a Sprague Dawley (SD) rat which has type I diabetes by administering streptozotocin (STZ) as confirmed by western blotting;

FIG. 2 shows an increase in expression of TENC1 protein in a fully differentiated podocyte cell line treated with a high concentration of glucose as confirmed by western blotting;

FIG. 3 shows induction of hypertrophy of podocyte cell lines by the protein tyrosine phosphatase (PTPase) activity of TENC1 when TENC1 WT and TENC1 phosphatase inactive mutant (CS) are overexpressed in differentiated podocyte cell lines using an adenovirus, as observed using a confocal microscope;

FIG. 4 shows activation of mTORC1 signaling which is confirmed by observing an increase in phosphorylation of S6K in differentiated podocyte cell lines treated with a high concentration of glucose;

FIG. 5A shows activation of mTORC1 signaling which is confirmed by observing an increase in phosphorylation of S6K depending on the concentration of treated TENC1 in a differentiated podocyte cell line, and FIG. 5B shows activation of mTORC1 induced TENC1 in a PTPase activity dependent manner;

FIG. 6 shows an immunoprecipitation result for confirming binding of TENC1 to nephrin phosphorylated by Fyn in a nephrin-overexpressing cell line (293NPH);

FIG. 7A shows decreases in nephrin phosphorylation and binding to PI3K when a differentiated podocyte cell line is treated with a high concentration of glucose, and FIG. 7B shows nephrin dephosphorylation induced by the PTPase activity of TENC1 when TENC1 WT or a TENC1 CS mutant is overexpressed after nephrin phosphorylation is induced by overexpressing Fyn in a nephrin-overexpressing cell line (293NPH);

FIG. 8 shows an inhibitory effect of the PTPase activity of TENC1 evaluated by a Malachite Green assay to screen a TENC1 inhibitor from a natural products library;

FIG. 9 shows that administration of DHTS, selected as the representative material of TENC1 inhibitors, to a db/db mouse which is a type II diabetic model does not affect a fasting serum glucose level;

FIG. 10 shows that renal hypertrophy observed in a db/db mouse is not observed in the kidney of the db/db mouse to which DI-ITS is administered, as detected by measuring a ratio of a kidney weight to a body weight of the mouse, to confirm the influence of the DHTS administration on the renal hypertrophy shown in diabetic nephropathy;

FIG. 11A shows that proteinuria increased as the development of diabetic nephropathy is decreased by DHTS administration in a db/db mouse, confirmed by albumin ELISA, and FIG. 11B shows that a damaged filtration function of podocytes caused by treating a differentiated podocyte cell line with a high concentration of glucose is improved by DHTS treatment, confirmed by protein quantification;

FIG. 12 shows that nephrin phosphorylation decreased in the kidney of a db/db mouse is recovered by DHTS administration, confirmed by immunoprecipitation; and

FIG. 13 shows that an increased activation of mTORC1 signaling is effectively improved by DHTS administration in a db/db mouse, as confirmed by western blotting.

### DETAILED DESCRIPTION OR EXEMPLARY EMBODIMENTS

From understanding that TENC1 having a PTPase activity is highly expressed in the diabetic kidney, the inventors have conducted research and thereby found that the dephosphorylation of nephrin, which is a podocyte protein, is mediated by TENC1 in diabetic nephropathy, leading to the completion of the present invention.

Thus, an aspect of the present invention provides a pharmaceutical composition comprising a TENC1 inhibitor as an active ingredient for preventing or treating diabetic nephropathy.

The TENC1 of the present invention may have the amino acid sequence of SEQ. ID. NO: 1, but the present invention is not limited thereto.

The term “diabetic nephropathy” used herein refers to a disease in which the glomerulus participating in blood filtration among the organs constituting the kidney is damaged due to diabetes and is a generic term of all diseases that can be caused by kidney function degradation due to the glomerular damage.

The term “prevention” used herein refers to all behaviors of inhibiting or delaying diabetic nephropathy by administration of a pharmaceutical composition according to the present invention.

The term “treatment” used herein refers to all behaviors involved in alleviating or beneficially changing symptoms of diabetic nephropathy by administration of a pharmaceutical composition according to the present invention.

According to an exemplary embodiment of the present invention, based on the result in which TENC1 is highly expressed in the diabetic kidney, it was confirmed that TENC1 is more highly expresses in kidney tissue of a SD rat with type I diabetes than normal kidney tissue, and TENC1 expression is increased when a podocyte known to be damaged from an early stage of the diabetic nephropathy is treated with a high concentration of glucose to create a high blood glucose environment, which is the main cause of diabetic nephropathy (see Example 1). It was experimentally proved that the TENC1 overexpressed under the high glucose condition substantially induced considerable podocyte hypertrophy, and such hypertrophy was caused by the PTPase activity of TENC1 (see Example 2).

In another exemplary embodiment of the present invention, relationship between an mTORC1 signaling mechanism known to be activated in the pathological mechanism of podocyte hypertrophy and TENC1 overexpressed in a high glucose environment was verified. The mammalian target of rapamycin complex 1 (mTORC1) regulates synthesis of a protein in response to insulin, a growth factor, an amino acid or oxidative stress, and the signaling is activated by S6K or eukaryotic initiation factor 4E (eIF4E)-binding protein 1 (4E-BP1), which is downstream of mTORC1. When mTORC1 is activated, at least two residues of S6K1 are phosphorylated, and protein synthesis is initiated. Therefore, it was confirmed that, when the podocyte cell line was treated with a high concentration of glucose, activation of mTORC1 was determined by an increase in S6K phosphorylation, and it was identified that the mTORC1 activation was caused by a PTPase activity of overexpressed TENC1 (see Example 3).

In still another exemplary embodiment of the present invention, from the result of verifying the influence of TENC1 on dephosphorylation of nephrin which is a podocyte-specific protein in a kidney damage environment such as diabetic nephropathy, it was confirmed that TENC1 was bound to nephrin phosphorylated by Fyn, phosphorylation of a tyrosine residue of nephrin was decreased in a high glucose environment causing the overexpression of TENC1, and dephosphorylation of the tyrosine residue of nephrin was substantially induced by the overexpression of TENC1 (see Example 4).

Based on the above results, in yet another exemplary embodiment of the present invention, screening using a natural compound library was performed to find a TENC1 inhibitor for inhibiting the activity of TENC1 which is capable of protecting podocytes by the inhibition of nephrin dephosphorylation. It was confirmed that four compounds among the library compounds exhibited an inhibitory effect of the PTPase activity of TENC1 and that a naphthoquinone structure was commonly found in the compounds (see Example 5).

In yet another exemplary embodiment of the present invention, DHTS was selected as the representative material from the compounds deduced by the screening, and an effect of DHTS in a diabetic nephropathy animal model was verified. As a result, it was observed that oral administration of DHTS does not change a fasting serum glucose level in a type II diabetic model, on the contrary, it was confirmed that the oral administration of DHTS has an effect of improving renal hypertrophy and proteinuria which are shown in diabetic nephropathy, and thus it was confirmed that such improvement is achieved by the inhibition of nephrin dephosphorylation and mTORC1 signaling activation caused by TENC1 (see Example 6).

Therefore, the TENC1 inhibitor of the present invention may be a naphthoquinone-based compound and is preferably one or more selected from the group consisting of (1R)-1,6-dimethyl-1,2-dihydronaphtho[1,2-g][1]benzofuran-10,11-dione (DHTS), (1R)-1,6,6-trimethyl-2,7,8,9-tetrahydro-1H-naphtho[1,2-g][1]benzofuran-10,11-dione (cryptotanshinone), 2,2-dimethyl-3,4-dihydrobenzo[h]chromene-5,6-dione (β-lapachone) and (4Z)-5-amino-6-(7-amino-6-methoxy-5,8-dioxoquinolin-2-yl)-4-(4,5-dimethoxy-6-oxocyclohexa-2,4-dien-1-ylidene)-3-methyl-1H-pyridine-2-carboxylic acid (streptonigrin), and more preferably, DHTS.

The compound derived from a natural product may be directly extracted from the natural product, and it is apparent to one of ordinary skill in the art that a chemically-synthesized compound also exhibits the same inhibitory effect of TENC1 activity as the compound extracted from the natural product.

The pharmaceutical composition of the present invention comprises a TENC1 inhibitor as an active ingredient and a pharmaceutically acceptable carrier. The pharmaceutically acceptable carrier is conventionally used in preparation and may be but is not limited to a saline solution, sterilized water, a Ringer's solution, a buffer, cyclodextrin, a dextrose solution, a maltodextrin solution, glycerol, ethanol, or liposome, etc., and, when needed, the composition of the present invention may further comprise a different conventional additive such as an antioxidant or a buffer solution. Also, the composition of the present invention may be prepared in an injectable form such as an aqueous solution, a suspension, an emulsion, etc., a pill, a capsule, a granule or a tablet by adding a diluent, a dispersant, a surfactant, a binder or a lubricant. Suitable pharmaceutically acceptable carriers may be prepared by a method disclosed in the Remington's Pharmaceutical Science. The pharmaceutical composition of the present invention may be prepared in the form of an injection, an inhalant, or an external preparation for skin, etc.

The pharmaceutical composition of the present invention may be used in oral administration or parenteral administration (e. g., intravenous, subcutaneous, dermal, nasal, or airway administration) according to a desired method, and a dose of the pharmaceutical composition may be dependent on the condition and body weight of a patient, severity of a disease, dosage form, or administration route and time, and may be suitably selected by those of ordinary skill in the art.

The composition of the present invention is administered at a pharmaceutically effective amount. The term “pharmaceutically effective amount” used herein refers to an amount sufficient to treat the disease at a reasonable benefit/risk ratio applicable for medical treatment, and an effective dosage may be determined by parameters including the type of illness of a patient, severity, drug activity, sensitivity to a drug, administration time, an administration route and a release rate, duration of treatment and co-used drugs, and other parameters well known in medical fields. The composition according to the present invention may be administered as a subject therapeutic agent, administered in combination with another therapeutic agent, sequentially or simultaneously administered with a conventional therapeutic agent, or administered in a single or multiple dose regimes. Taking all of the factors into consideration, it is important to achieve the maximum effect with the minimum dose without a side effect, and such a dose may be easily determined by one of ordinary skill in the art.

Specifically, the effective amount of the compound according to the present invention may vary depending on age, sex or body weight of a patient, and the compound may be generally administered at 0.001 to 150 mg and, preferably, 0.01 to 100 mg per kg of body weight, daily, every other day, or once to three times a day. However, the effective amount may vary depending on the administration route, severity of obesity, sex, body weight or age, and therefore, it should be noted that the scope of the present invention is not limited by the dose by any means.

Another aspect of the present invention provides a method for preventing or treating diabetic nephropathy including administering the composition to a subject.

The teem “subject” used herein refers to a target with a disease to be treated and, more specifically, a mammal such as a human or non-human primate, a mouse, a rat, a dog, a cat, a horse or a cow.

Also, the present invention provides a use of the composition for preventing or treating diabetic nephropathy.

Hereinafter, exemplary examples will be provided to help in understanding of the present invention. However, the following examples are merely provided to more easily understand the present invention, and the scope of the present invention is not limited by the following examples.

### EXAMPLES

### Example 1

### Confirmation of Increased TENC1 Expression in Diabetic Nephropathy Environment

1-1. Confirmation of Increased TENC1 Expression in Kidney Tissue of Diabetic Model

According to previous research, the inventors confirmed that TENC1 is involved in muscle atrophy in a diabetic environment and confirmed from a database that TENC1 is more highly expressed in the kidney than other organs. Therefore, based on the above results, it was investigated whether TENC1 is associated with diabetic nephropathy which is a major part of the complications caused by diabetes.

To verify the relationship between TENC1 and diabetic nephropathy, type I diabetes was induced by administering a high dose of streptozotocin (hereinafter, referred to as STZ) which is a compound widely used in manufacturing a type I diabetic animal model by inducing cytotoxicity to beta cells of the pancreas producing insulin, to a SD rat. The kidney was extracted from the diabetic SD rat and subjected to western blotting to identify an expression level of TENC1 protein.

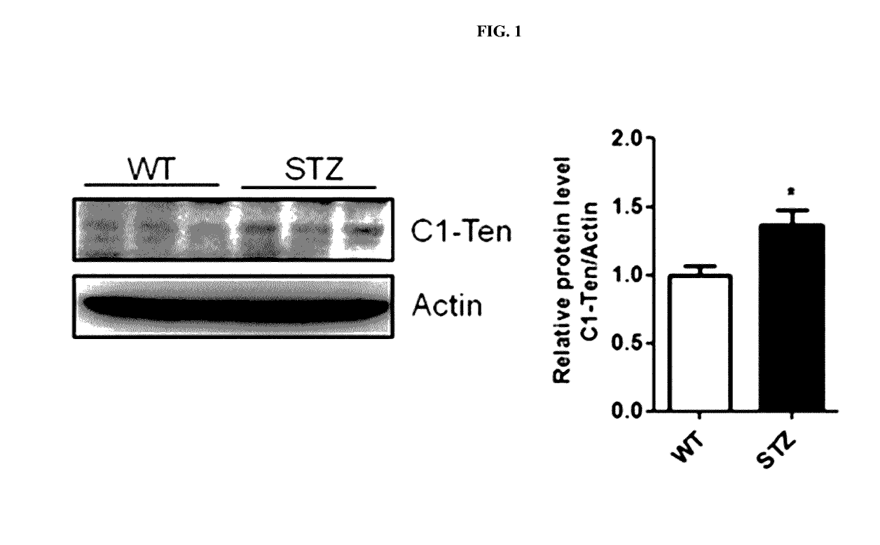

As a result, referring to FIG. 1, it was seen that, compared with renal cells of a SD rat in which diabetes is not induced, a statistically significant increase in TENC1 (C1-Ten) expression is shown in renal cells of the SD rat to which diabetes is induced by STZ.

1-2. Confirmation of Increase in TENC1 Expression in Podocyte Cell Line Treated with High Glucose

From the result of Example 1-1, it was confirmed that TENC1 expression is increased in kidney tissue of the diabetic SD rat, and then it was investigated that the relationship between TENC1 and podocytes which is known to have an important role in an early development of diabetic nephropathy. Since a high blood glucose level increased in a diabetic environment plays a critical role in various mechanisms involved in diabetic nephropathy, a differentiated podocyte cell line was treated with glucose at a high concentration of 30 mmol/L and subjected to western blotting for identifying an expression level of TENC1 protein.

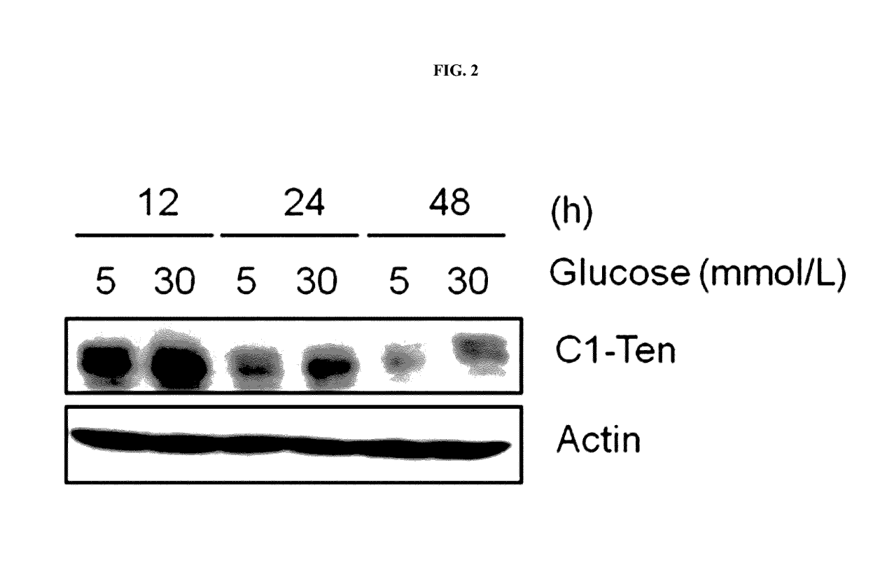

As a result, referring to FIG. 2, when the podocyte cell line was treated with a high concentration of glucose for 12, 24 or 48 hours, compared with when treated with glucose at a normal concentration, that is, 5 mmol/L, TENC1 expression was increased.

From the above result, it was seen that TENC1 is associated with the pathological symptom of the podocytes exhibited along with the diabetic nephropathy.

### Example 2

### Confirmation of Induction of Hypertrophy of Podocyte Cell Line by TENC1 Overexpression

Based on the result of Example 1, an experiment that will be described below was performed to check which pathological symptom is substantially exhibited in the kidney by TENC1 increased in expression in a podocyte cell line treated with a high concentration of glucose.

A podocyte cell line expressing green fluorescent protein (GFP) and TENC1 proteins was prepared by infecting a differentiated podocyte cell line by an adenovirus into which GFP and TENC1 expression vector are inserted. TENC1 was overexpressed in a podocyte cell line using TENC1 wild type (WT) or TENC1 mutant (TENC1 CS) manufactured to verify that a TENC1 effect is caused by the PTPase activity of TENC1 as identified from the previous research by substituting cysteine at amino acid position 231 of TENC1 protein by serine. The TENC1 CS is a mutant which is manufactured to stably bind to a substrate, but not to have a catalytic action. Afterward, the size of the differentiated podocyte cell line was observed under a confocal microscope using green fluorescence generated by GFP expression.

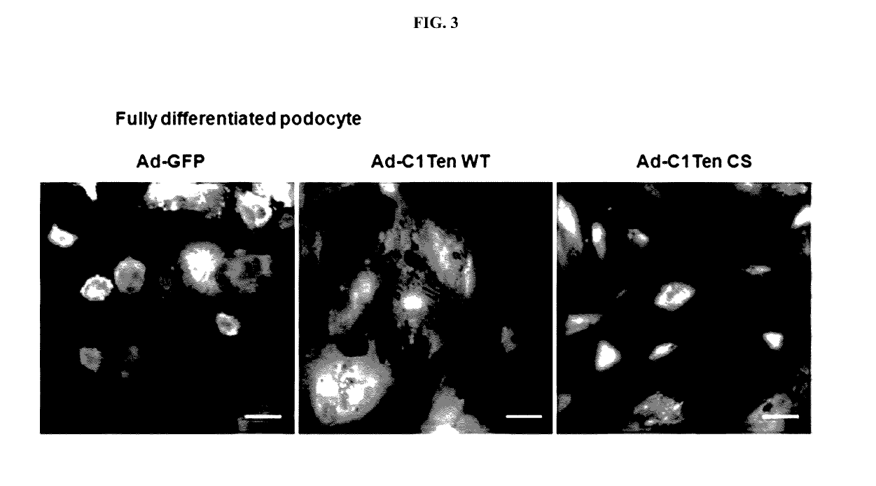

As a result, referring to FIG. 3, it was seen that TENC1 WT-overexpressing cells (Ad-C1 Ten WT) were considerably larger than GFP only-expressing cells (Ad-GFP). Also, it was confirmed that TENC1 mutant-overexpressing cells (Ad-C1 Ten CS) had a size similar to only GFP-expressing cells. Therefore, from the result, it was seen that TENC1 can induce hypertrophy of podocytes in a PTPase dependent manner.

### Example 3

### Confirmation of Influence of TENC1 on mTORC1 Signaling Mechanism

Podocyte hypertrophy is one of the main characteristics of damaged podocytes generated along with the death and detachment of podocytes in the early diabetic nephropathy. Also, it has been reported that mTORC1 is activated and serves as the most important regulator in a pathological mechanism of the podocyte hypertrophy. Therefore, it was investigated whether the treatment of high concentration of glucose induces mTORC1 activation in a differentiated podocyte cell line.

To this end, following treatment of the differentiated podocyte cell line with glucose at a concentration of 5, 11.1, or 30 mmol/L for 48 hours, to confirm mTORC1 activation, an increase in tyrosine phosphorylation of P70-S6 kinase 1 (S6K) whose expression is stimulated by mTORC1 signaling activation was identified by western blotting.

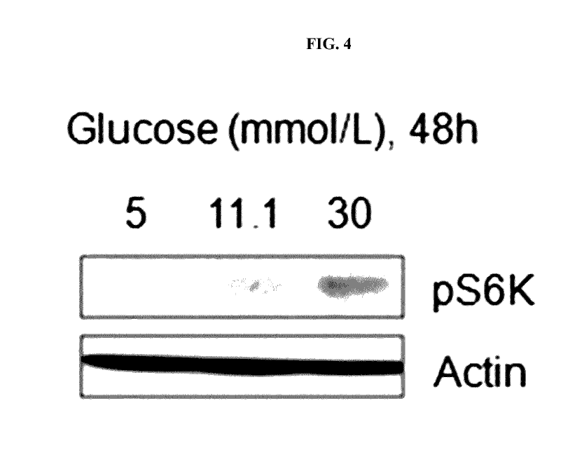

As a result, referring to FIG. 4, it can be seen that stimulation of S6K phosphorylation (pS6K) in the differentiated podocyte cell line depends on glucose concentration.

Also, according to the result of Example 1-2, it was seen that, when the differentiated podocyte cell line was treated with a high concentration of glucose, the expression of TENC1 was increased, and therefore whether the increase in TENC1 expression is involved in the activation of mTORC1 signaling was examined. Therefore, in the same manner as described in Example 2, GFP and TENC1 WT were overexpressed in differentiated podocytes using an adenovirus vector, and S6K phosphorylation was identified. Further, to verify whether a change in mTORC1 signaling by TENC1 is caused by the PTPase activity of TENC1, TENC1 CS was overexpressed along with GFP using an adenovirus vector, and an amount of S6K phosphorylation was evaluated.

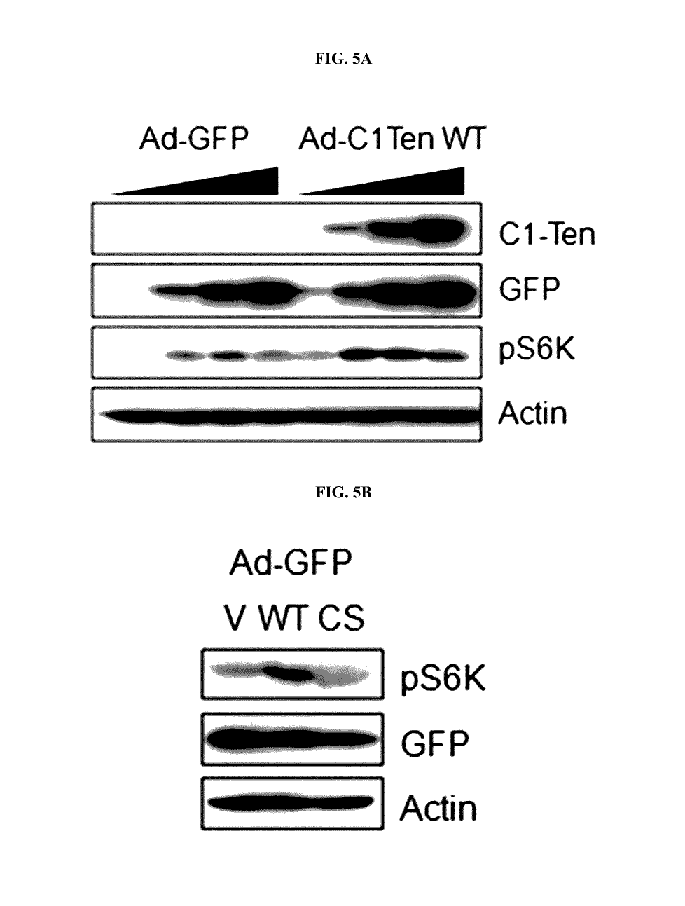

As a result, referring to FIG. 5A, compared with a control podocyte cell line only expressing GFP, it was confirmed that S6K phosphorylation (pS6K) was increased in a TENC1 WT-overexpressed podocyte cell line. However, referring to FIG. 5B, in the podocyte cell line overexpressing TENC1 CS, S6K phosphorylation was not observed.

Therefore, it was confirmed that the podocyte hypertrophy can be caused by mTORC1 signaling activated by TENC1 in a PTPase dependent manner.

### Example 4

### Confirmation of Mechanism of Inhibiting Nephrin Phosphorylation by TENC1 in Podocytes

4-1. Confirmation of Binding Between TENC1 and Nephrin Through Immunoprecipitation

Recent research showed that nephrin, a podocyte-specific protein, can be phosphorylated by Fyn and dephosphorylated in a kidney damage environment such as diabetic nephropathy. However, despite the importance, there is not enough research on downstream signaling in a podocyte via nephrin phosphorylation and the regulator thereof. However, since it was confirmed from the results of the examples that the PTPase activity of TENC1 can play a major role in the pathological mechanism of podocytes, in this example, it was investigated whether TENC1 is involved in inhibiting the nephrin phosphorylation.

To this end, first, a nephrin-overexpressing cell line (293NPH) was prepared using a HEK293, nephrin was phosphorylated by overexpressing Fyn known to phosphorylate nephrin in the prepared cell line, and then TENC1 WT or TENC1 CS was overexpressed to investigate whether TENC1 binds to nephrin through immunoprecipitation (IP).

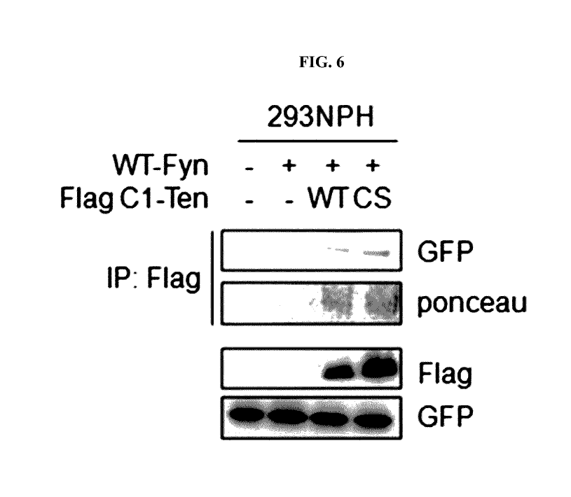

Consequently, referring to FIG. 6, it can be seen that, while Fyn is overexpressed all of overexpressed TENC1 WT and TENC1 CS bind to TENC1-phosphorylated nephrin.

4-2. Confirmation of Inhibition of Nephrin Phosphorylation by TENC1 in Podocytes

Because it was confirmed that TENC1 binds to nephrin phosphorylated by Fyn in Example 4-1, it was further investigated whether TENC1 inhibits nephrin phosphorylation.

To this end, first, to examine whether nephrin phosphorylation is inhibited in a high glucose environment that can cause TENC1 overexpression, the differentiated podocyte cell line was treated with glucose at 5 mmol/L or 30 mmol/L for 12 hours or 24 hours, respectively, and then subjected to immunoprecipitation (IP) and immunoblotting.

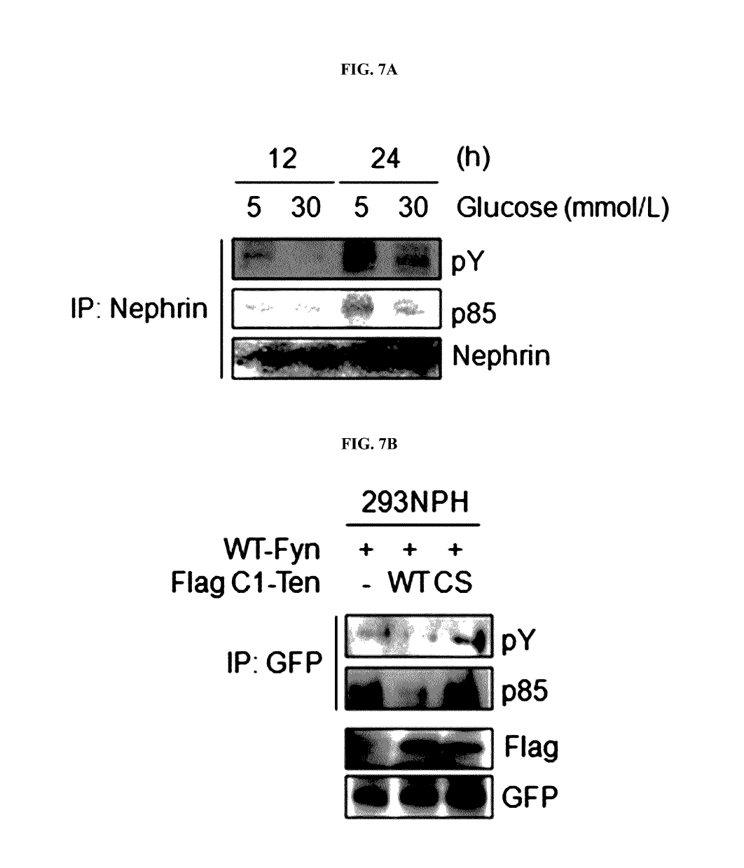

Consequently, as shown in FIG. 7A, when the cells were treated with glucose at a high concentration of 30 mmol/L, compared with when treated with 5 mmol/L of glucose, tyrosine residue phosphorylation (pY) of nephrin was reduced. Also, by examining p85, which is a regulatory subunit of PI3K, to evaluate an amount of binding to PI3K known to bind when nephrin is phosphorylated, it was seen that the binding to PI3K was also reduced when a high concentration of glucose was treated.

Subsequently, to verify whether nephrin phosphorylation is directly inhibited by TENC1, after overexpression of Fyn to induce the nephrin phosphorylation, in the nephrin-overexpressing podocyte cell line (293NPH) prepared in Example 4-1, TENC1 was not expressed or TENC1 WT or TENC1 CS was overexpressed and then immunoprecipitation (IP) was performed.

As a result, referring to FIG. 7B, it was confirmed that, when TENC1 WT was overexpressed, the nephrin dephosphorylation was induced compared with the control, and the binding between nephrin and PI3K was reduced. However, it was seen that, when the TENC1 mutant (TENC1 CS) was overexpressed, the nephrin dephosphorylation is not observed and the binding to PI3K is not reduced, either.

According to the above results, it was confirmed that, as nephrin serves as a substrate of TENC1, the nephrin phosphorylation is regulated by TENC1, particularly, the PTPase activity of TENC1, and increased TENC1 expression due to the high glucose environment also may serve to inhibit the nephrin phosphorylation in a high glucose environment.

### Example 5

### Finding TENC1 Inhibitor by Screening

According to the above results, it was confirmed that TENC1 overexpressed in a high glucose environment known as a leading cause of diabetic nephropathy inhibits phosphorylation of nephrin caused by dephosphorylation, and induces hypertrophy of the podocyte cell line. For this reason, to inhibit the hypertrophy of the podocyte cell line, screening was performed through a Malachite Green assay for finding a TENC1 inhibitor.

TENC1 protein isolated from the cells and each of the natural product library candidate compounds were put together for a predetermined time, and the PTPase activity of TENC1 was measured. If the PTPase activity was reduced compared with the control, it can be considered that the added candidate compound inhibited the TENC1 activity. Here, as a positive control, a PTPase inhibitor, sodium vanadate (Na₃VO₄; NAV) was used. Since there were almost no molecules left in the test tube, the method has a higher probability of discovering a direct regulator, compared with a cellular-level screening method.

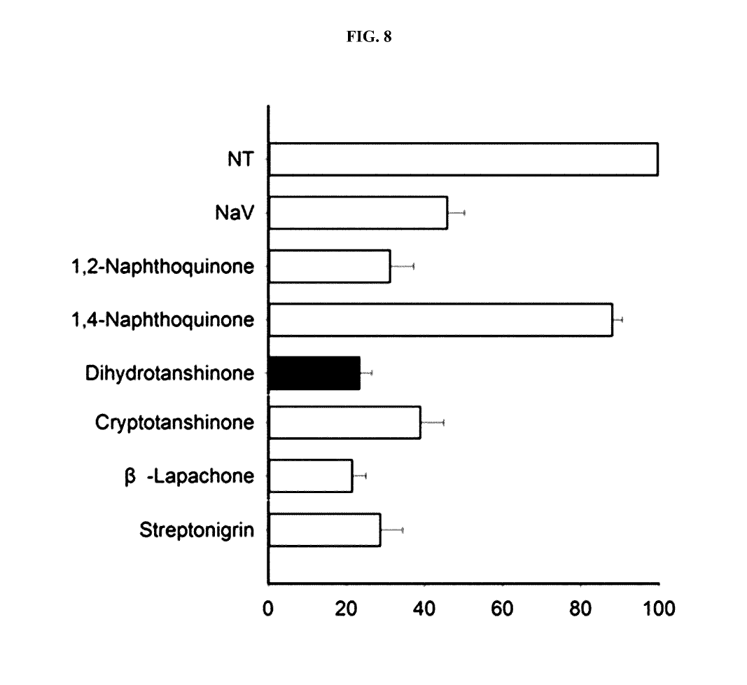

Consequently, referring to FIG. 8, it was confirmed that four types of natural products having a naphthoquinone structure including DHTS has an inhibitory effect on the PTPase activity of TENC1. Since TENC1 can be a target for diabetic nephropathy, the TENC1 inhibitor can be used as a therapeutic agent for diabetic nephropathy.

### Example 6

### Analysis of Effect of TENC1 Inhibitor in Diabetic Nephropathy Animal Model

An effect of DHTS, which is the representative material of the TENC1 inhibitors found through the screening described in Example 5, was identified using a diabetic nephropathy animal model. To this end, as a diabetic nephropathy animal model, a type II diabetic model, that is, a db/db mouse was used, and as a control, a db/m mouse was used. 10 week-old male db/m and db/db mice were divided into three groups including a group in which a vehicle was administered to a db/m mouse, a group in which a vehicle was administered to a db/db mouse, and a group in which 300 mg/kg of DHTS was administered to a db/db mouse for experiments, and the vehicle or drug was orally administered daily for 2 weeks according to each condition. As the vehicle, 1% carboxymethyl cellulose was used.

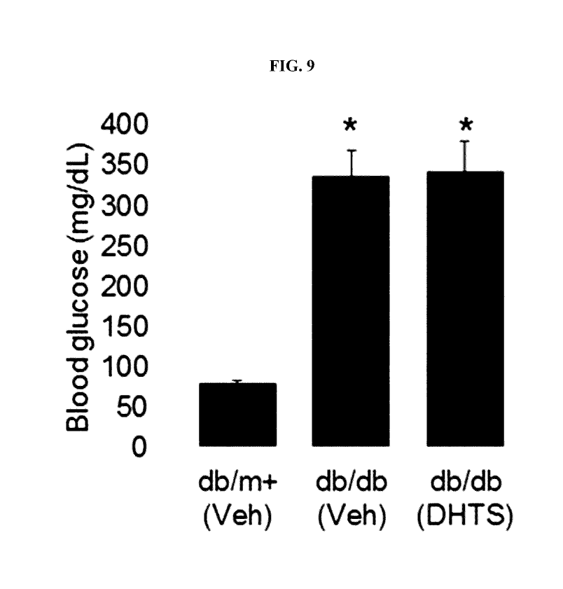

First, after the experiment described above, blood was taken from a mouse to measure a fasting serum glucose level. As a result, referring to FIG. 9, it was confirmed that the fasting serum glucose level was not decreased by the administration of DHTS for two weeks.

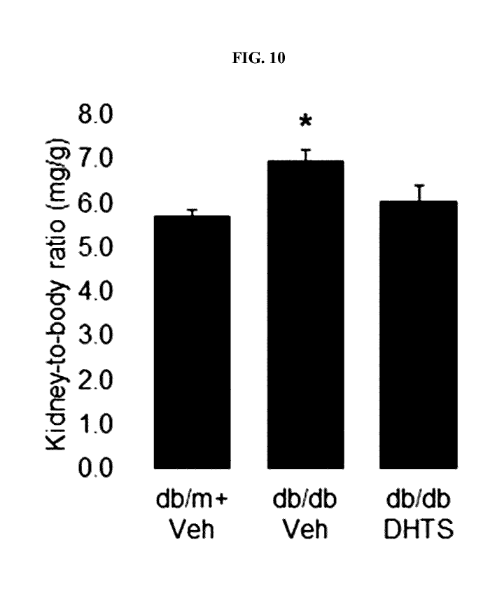

Subsequently, to examine whether DHTS can exhibit a kidney protective effect due to the inhibition of TENC1 PTPase regardless of the decrease of a blood glucose level, a mouse was sacrificed, the kidney was extracted and weighed to investigate whether renal hypertrophy shown in diabetic nephropathy can be improved, and a urea sample of the mouse was obtained to investigate whether proteinuria shown in diabetic nephropathy can be improved. Consequently, as shown in FIG. 10, the kidney weight of the db/db mouse, which is the vehicle-injected diabetic model, was higher than that of the db/m mouse, and such an increase was not shown in the kidney of the DHTS-injected db/db mouse. Therefore, it was confirmed that DHTS can improve the renal hypertrophy.

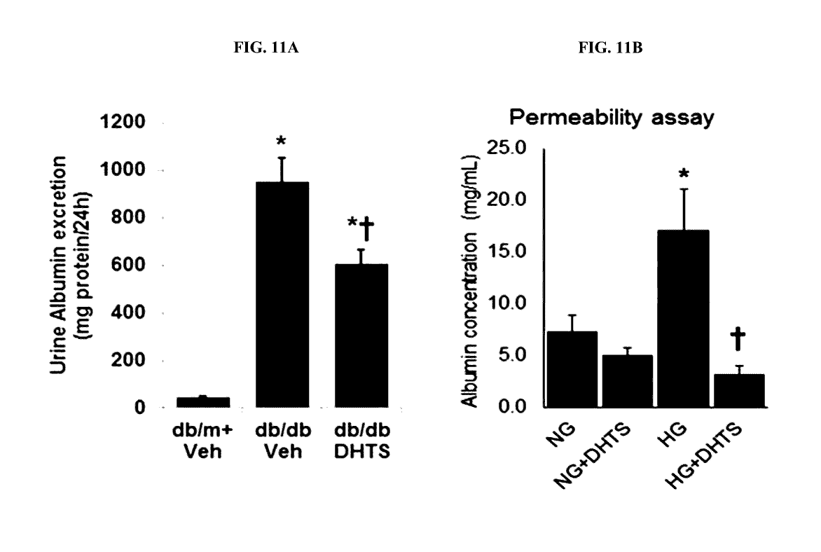

Also, referring to FIG. 11A, it was confirmed that proteinuria was significantly reduced in the DHTS-injected db/db mouse, compared with the vehicle-injected db/db mouse. Moreover, referring to FIG. 11B, it was confirmed that, even when a filtration function of the podocyte was damaged by providing a high glucose condition of 30 mmol/L to the differentiated podocyte, because of the treatment with DHTS, the damaged filtration function can be improved to a level similar to the result obtained under a the normal glucose condition of 5 mmol/L.

Further, to investigate whether the above result is obtained by inhibiting TENC1-induced nephrin dephosphorylation and increased mTORC1 signaling, immunoprecipitation and western blotting were performed with a kidney tissue sample extracted from a mouse.

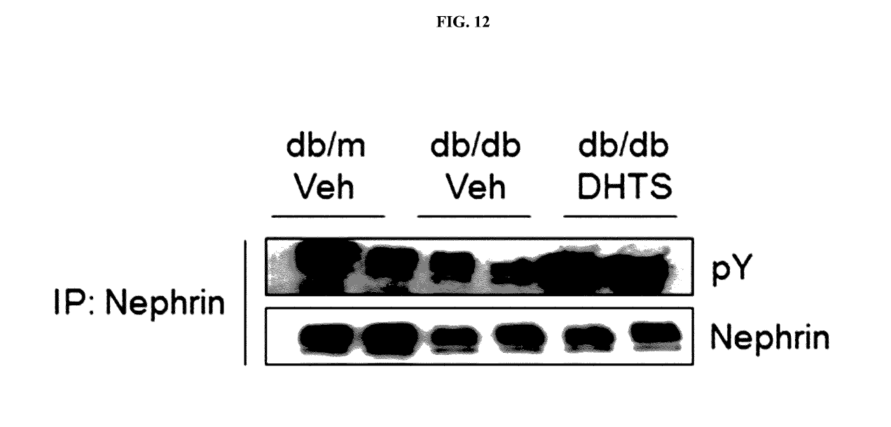

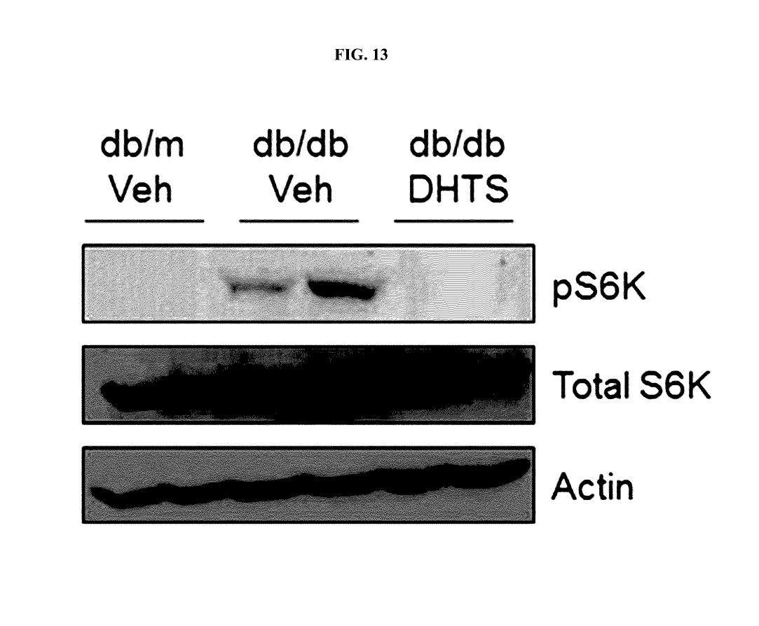

As a result, referring to FIG. 12, it was confirmed that nephrin dephosphorylation is shown in the kidney tissue of a db/db mouse compared with a db/m mouse, and nephrin phosphorylation is recovered in the kidney tissue of a DHTS-oral administered db/db mouse. Also, referring to FIG. 13, it was confirmed that S6K phosphorylation (pS6K) is increased in kidney tissue of the db/db mouse compared with the db/m mouse, but the increase in S6K phosphorylation (pS6K) is effectively inhibited by oral administration of DHTS. According to the above results, it was confirmed that nephrin dephosphorylation and increases in mTORC1 signaling shown in the kidney tissue of a db/db-induced type II diabetic nephropathy model are improved by the representative material of the TENC1 inhibitor, DHTS and thus renal hypertrophy and proteinuria shown in diabetic nephropathy are also improved.

It would be understood by those of ordinary skill in the art that the above descriptions of the present invention are exemplary, and the example embodiments disclosed herein can be easily modified into other specific forms without changing the technical spirit or essential features of the present invention. Therefore, it should be interpreted that the example embodiments described above are exemplary in all aspects, and are not limitative.

What is claimed is:

1. A method for preventing or treating diabetic nephropathy, comprising:
administering a composition comprising tensin-like C1 domain-containing phosphatase (TENC1) inhibitor as an active ingredient to a subject.

2. The method of claim 1, wherein the TENC1 inhibitor is a naphthoquinone-based compound.

3. The method of claim 2, wherein the naphthoquinone-based compound is one or more selected from the group consisting of (1R)-1,6-dimethyl-1,2-dihydronaphtho[1,2-g][1]benzofuran-10,11-dione (dihydrotanshinone; DHTS), (1R)-1,6,6-trimethyl-2,7,8,9-tetrahydro-1H-naphtho[1,2-g][1]benzofuran-10,11-dione (cryptotanshinone), 2,2-dimethyl-3,4-dihydrobenzo[h]chromene-5,6-dione (β-lapachone), and (4Z)-5-amino-6-(7-amino-6-methoxy-5,8-dioxoquinolin-2-yl)-4-(4,5-dimethoxy-6-oxocyclohexa-2,4-dien-1-ylidene)-3-methyl-1H-pyridine-2-carboxylic acid (streptonigrin).

4. The method of claim 3, wherein the naphthoquinone-based compound is DHTS.

5. The method of claim 1, wherein the TENC1 comprises the amino acid sequence of SEQ. ID. NO: 1.

6. The method of claim 1, wherein the TENC1 inhibitor inhibits the protein tyrosine phosphatase activity of TENC1.

7. The method of claim 1, wherein the composition has a podocyte protective effect by inhibiting nephrin dephosphorylation induced by TENC1.

8. The method of claim 1, wherein the composition further comprises a pharmaceutically acceptable carrier or additive.
